(19) United States
(12) Patent Application Publication
(10) Pub. No.: US 2004/0092754 A1
(43) Pub. Date: May 13, 2004

(54) Derivatives of 4-(trifluoromethyl)-phenol and 4-(trifluoromethylphenyl)-2-(tetrahydropyranyl) ether and method for producing the same

(76) Inventors: Bernd Schafer, Dierbach (DE); Herve Geneste, Neuhofen (DE)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/344,612

(22) PCT Filed: Aug. 9, 2001
(86) PCT No.: PCT/EP01/09222

(30) Foreign Application Priority Data
Aug. 11, 2000 (DE) 10040051.5

(51) Int. Cl. C07F 5/02; C07C 323/16; C07C 65/03; C07C 49/245; C07C 39/24; C07F 7/04

(52) U.S. Cl. 549/416; 556/449; 562/7; 562/475; 568/62; 568/429; 568/333; 568/775

(57) ABSTRACT

The invention relates to a method for producing 2- and 2,5-substituted derivatives of 4-(trifluoromethyl)-phenol and 4-(2-trifluoromethyl)-phenyl)-2-tetrahydropyranyl) ether and to novel derivatives. Said method is characterised in that a compound of formula (2) 4-(trifluoromethylphenyl)-2-(tetrahydropyranyl) ether is reacted with an electrophile E-X or a combination of electrophiles E-X and E-Y in the presence of a base, X and Y having the meanings given in the description.

### INTRODUCTION

The invention is in the field of the derivatives of 4-(trifluoromethyl)phenol and of the derivatives of 4-(trifluoromethyl)phenyl 2-tetrahydropyranyl ether and relates to novel derivatives and to a process for the preparation of these derivatives.

### PRIOR ART

In WO 98/29411, the synthesis of 2-hydroxy-5-trifluoromethyl-benzaldehyde by reaction of 4-(trifluoromethyl)phenol with hexamethylenetetramine is described. In addition to the unsatisfactory yield (43%), the long reaction time (24 h), the use of corrosive and toxic trifluoroacetic acid and the chromatographic purification necessary are disadvantageous. WO 98/42664 describes the synthesis of 2-hydroxy-5-trifluoromethyl-benzaldehyde by reaction of 4-(trifluoromethyl)phenol with tin tetrachloride and paraformaldehyde at 110° C. and a reaction time of 12 hours. In addition to the low yield (12%), the chromatographic purification of the product necessary and the use of toxic tin chloride and mutagenic paraformaldehyde are also disadvantageous here.

The synthesis of 2-methoxy-5-trifluoromethyl-benzaldehyde by reaction of p-trifluoromethylanisole with butyllithium and N-formylpiperidine is described by K. K. Laali, G. F. Koser, S. Subramanyam, D. A. Forsyth, J. Org. Chem. 58 (1993) 1385. The yield is only 41%.

The synthesis of 2-hydroxy-5-trifluoromethylacetophenone by photolysis (photo-Fries rearrangement) of 4-(trifluoromethyl)-phenol in the presence of 3,3-dimethyl-2-butanone (pinacolone) or 1,1,1-trichloroethane is described in M. A. Miranda, J. Chem. Soc., Chem. Commun. (1995) 2009; F. Galindo, M. C. Jiménez, M. A. Miranda, R. Tormos, J. Photochem. Photobiol., A 97 (1996) 151 and H. Garcia, Synthesis (1985) 901. However, the conversion is only 25%, a reaction product being obtained which contains 63% of 2-hydroxy-5-trifluoromethylacetophenone.

DE 2653601 A1 describes the preparation of 2-hydroxy-5-trifluoro-methylacetophenone via Friedel-Crafts acylation of 4-(trifluoromethyl)phenol with carboxylic acid halides. However, this reaction necessitates the use of anhydrous hydrogen fluoride, which is very corrosive and extremely toxic.

DE 2907379 describes the synthesis of aromatic ketones (R =H, X =CO(aryl)) in a Grignard reaction from the O-methyl-protected 2-cyano-4-(trifluoromethyl)phenol. This synthesis route has the following disadvantages: 2-cyano-4-(trifluoromethyl)phenol is only accessible via many reaction steps. The preparation of Grignard reagents harbors the danger of accumulation of the starting material. The methyl protective group must then be removed again in an individual reaction step using boron trichloride, which is very expensive.

FR 2735771 describes the synthesis of 2-hydroxy-5-trifluoro-methylbenzoic acid from the potassium salt of 4-(trifluoromethyl)phenol via Kolbe synthesis. The reaction necessitates a reaction temperature of 180° C. and a carbon dioxide overpressure of 10 bar. This necessitates special apparatus. Moreover, the yield is only 45%. An alternative preparation is described by S. Yamamoto, S. Hashiguchi, S. Miki, Y. Igata, T. Watanabe, M. Shiraishi, Chem. Pharm. Bull. 44 (1996) 734; starting from methyl 5-iodo-2-methoxybenzoate, this is reacted at 160° C. with sodium trifluoroacetate in the presence of copper iodide. The methyl groups are removed using boron tribromide. The yield over both steps is 16%. A further synthesis route is described by J. Alexander, Org. Prep. Proced. Int. 18 (1986) 213: there, 3-bromo-4-(trifluoromethyl)phenol is methylated using dimethyl sulfate, lithiated using butyllithium, reacted with carbon dioxide and finally demethylated using pyridinium chloride. The synthesis route via the bromo compound and the protection of the hydroxyl group with a methyl group which has to be removed again in a separate reaction step is cumbersome. In U.S. Pat. No. 5,712,279, 4-(trifluoromethyl)phenol is finally reacted with diethylcarbamyl chloride, carboxylated at −73° C. using s-butyllithium/carbon dioxide and the protective group is then removed with citric acid. Working with s-butyllithium, in contrast to n-butyllithium, is not unproblematical. Furthermore, the use of diethylcarbamyl chloride is undesirable for toxicological reasons (carcinogenic, causes hereditary damage).

EP 206951 A1 describes the synthesis of 2-chloro-4-(trifluoro-methyl)phenol by trifluoromethylation of 2-chlorophenol, and J. C. Blazejewski, R. Dorme, C. Wakselman, J. Chem. Soc., Perkin Trans. 1 (1987) 1861 describe the synthesis of 2-chloro-4-(trifluoro-methyl)phenol by reaction of 4-(trifluoromethyl)phenol with sodium chlorite and sulfuric acid. In both cases, the yield is below 10% and the product is contaminated with a large number of other components. Direct chlorination of 4-(trifluoro-methyl)phenol is described in EP 0188031 B1, DE 2311638 C1 and CH 567359. The nucleophilic aromatic substitution of a chlorine of 1,2-dichloro-4-(trifluoromethyl)phenol also leads to the desired product. However, drastic conditions (potassium hydroxide in DMSO) and long reaction times (47 h) are necessary for the reaction in order to achieve good yields (90%) (FR 2529197, EP 19388, JP 59139336, U.S. Pat. No. 4,548,640).

The preparation of 2-bromo-4-(trifluoromethyl)phenol by direct bromination of 4-(trifluoromethyl)phenol with elemental bromine is described (S. Yamamoto, S. Hashiguchi, S. Miki, Y. Igata, T. Watanabe, M. Shiraishi, Chem. Pharm. Bull. 44 (1996) 734; WO 9749710, EP 477789, EP 49383). Elemental bromine is toxic and corrosive. Working with this substance necessitates considerable safety measures.

JP 57136591 describes the use of 2-methylmercapto-4-(trifluoro-methyl)phenol (R =H, X =SR¹).

4-(Trifluoromethyl)-1,2-benzenediol is obtained by diazotization and boiling of 2-amino-4-(trifluoromethyl)phenol. The yield is 45% (J. P. Chupp, C. R. Jones, M. L. Dahl, J. Heterocycl. Chem. 30 (1993) 789).

A large number of methods for the synthesis of individual 2-substituted derivatives of 4-(trifluoromethyl)phenol have been described in the prior art. In all known processes, the reaction times, which are long in some cases, the low yields and the use of toxicologically harmful chemicals are disadvantageous. In a number of the known processes, a complicated chromatographic purification is necessary in order to obtain the reaction product.

The 2,5-disubstituted derivatives of 4-(trifluoromethyl)phenol and the 2-substituted and. the 2,5-disubstituted derivatives of 4-(trifluoromethyl)phenyl 2-tetrahydropyranyl ether and processes for their preparation are not described in the prior art.

It is an object of the present invention to develop a process which makes it possible to prepare a large number of 2- and 2,5-substituted derivatives of 4-(trifluoromethyl)phenol and 2- and 2,5-substituted derivatives of 4-(trifluoromethyl)phenyl 2-tetrahydropyranyl ether in high yield. At the same time, a simple and rapid procedure and low demands on safety equipment were desirable. In particular, handling of toxicologically harmful chemicals should be avoided. Furthermor , tedious purification of the product from the reaction mixture should be avoided. Ideally, it should be possible to employ the products directly, without further purification, for further reactions. A further object was the preparation of novel, previously unknown 2- and 2,5-disubstituted derivatives of 4-(trifluoro-methyl)phenol and of 2- and 2,5-disubstituted derivatives of 4-(trifluoro-methyl)phenyl 2-tetrahydropyranyl ether.

### DESCRIPTION

It has been found that using the process according to the invention these compounds can be prepared in a simple manner and with a high yield. The use of toxicologically harmful chemicals is avoided and the requirements in terms of apparatus are low.

The present invention therefore relates to a process for the preparation of compounds of the general formula (1)

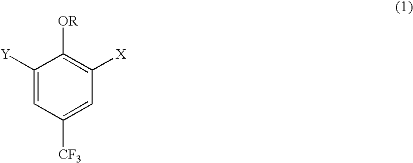

where

R is hydrogen, tetrahydropyran-2-yl,

X is B(OH)₂, B(OR¹)₂, B(Oaryl)₂, B(OR¹)(Oaryl), CHO, COR¹, CO(aryl), COOH, COOR¹, SiR¹₃, Si(aryl)₃, OH, SR¹, S(aryl), Cl, Br, I,

Y is H, B(OH)₂, B(OR¹)₂, B(Oaryl)₂, B(OR¹)(Oaryl), CHO, COR¹, CO(aryl), COOH, COOR¹, SiR¹₃, Si(aryl)₃, OH, SR¹, S(aryl), Cl, Br, I,

R¹ independently of one another is C₁-C₈-alkyl,

aryl independently of one another is phenyl, naphthyl, R¹-C₆H₄, R¹O-C₆H₄,

which comprises reacting the compound of the formula 2 4-(trifluoromethyl)phenyl 2-tetrahydropyranyl ether

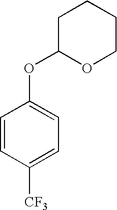

in the presence of a base with an electrophile E-X or a combination of electrophiles E-X and E-Y, where X and Y have the meanings indicated above.

Essential to the invention is the use of 4-(trifluoromethyl)phenyl 2-tetrahydropyranyl ether; it makes possible targeted derivatization in the 2 or in the 2 and 5 positions.

Using this process, both the monosubstituted (Y =H) derivatives and the disubstituted derivatives (Y≠H) are accessible. In a preferred embodiment, monosubstituted derivatives are prepared. In the case of the disubstituted derivatives (substitution in the 2 and 5 positions), both the symmetrical derivatives (X and Y identical) and the unsymmetrical derivatives (x different from Y) can be prepared. The preparation of the symmetrical derivatives is particularly preferred.

The corresponding derivatives of 4-(trifluoromethyl)phenol are accessible by removal of the tetrahydropyranyl protective group after substitution has taken place.

The compound of the formula (2) [4-(trifluoromethyl)phenyl 2-tetrahydropyranyl ether] is known; it is accessible synthetically as described, for example, by A. Ouedraogo, J. Lessard, Can. J. Chem. 69 (1991) 474.

Depending on the final product to be synthesized, the electrophiles E-X and/or E-Y employed are compounds in which E is a good leaving group and X and/or Y inserts the desired side chain.

For the preparation of derivatives in which X =CHO, a suitable electrophile E-X is the compound of the general formula X-NR¹₂ (3)

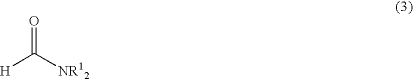

For the preparation of derivatives in which X =COR¹ or X =CO(aryl), suitable electrophiles E-X are compounds of the general formula X-N(CH₃)(OCH₃) (4) and (5).

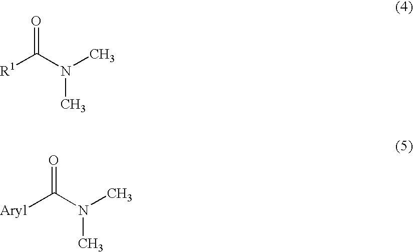

For the preparation of derivatives in which X =COOR₁, suitable electrophiles E-X are compounds of the general formula (6) X-OR¹.

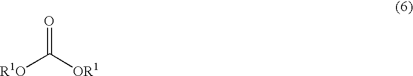

For the preparation of derivatives in which X =COOH, a suitable electrophile is carbon dioxide in gaseous or solid form.

For the preparation of derivatives in which X =Cl, Br, I, a suitable electrophile is chlorine, bromine, iodine and/or appropriate compounds of the general formulae (7) and/or (8) and/or (9)

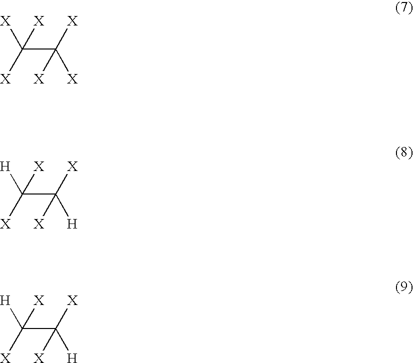

For the preparation of derivatives in which X =SR¹, suitable electrophiles E-X are compounds of the general formula (10): X-SR¹ (=R¹S-SR¹).

For the preparation of derivatives in which X =S(aryl), suitable electrophiles are compounds of the general formula (11):

X-S(aryl) (=(aryl)S-S(aryl)).

For the preparation of derivatives in which X =B(OR¹)₂, suitable electrophiles are compounds of the general formula (12): X-B(OR¹)₂ (=B(OR¹)₃) or a mixture of BF₃ or BCl₃ and compounds of the general formula (12).

For the preparation of derivatives in which X =B(OH)₂, suitable electrophiles are compounds of the general formula (12) or a mixture of BF₃ and (12) and subsequent addition of water.

For the preparation of derivatives in which X =OH, suitable electrophiles are compounds of the general formula (12) or a mixture of BF₃ and compounds of the general formula (12) and subsequent addition of hydrogen peroxide.

For the preparation of derivatives in which X =B(Oaryl)₂, suitable electrophiles E-X are compounds of the general formula (13): X-B(Oaryl)₂ (=B(Oaryl)₃) or a mixture of BF₃ or BCl₃ and compounds of the general formula (13).

For the preparation of derivatives in which X =SiR¹₃, suitable electrophiles E-X are compounds of the general formula (14): X-Cl (=R¹₃SiCl).

For the preparation of derivatives in which X =Si(aryl)₃, suitable electrophiles E-X are compounds of the general formula (15): X-Cl (=(aryl)₃SiCl).

For the preparation of derivatives in which X =B(OR¹)(Oaryl), suitable electrophiles E-X are compounds of the general formula (16) B(OR¹)(Oaryl)₂ or of the general formula (17) B(OR¹)₂(Oaryl).

For the unsymmetrical disubstituted compounds, the corresponding mixtures of the electrophiles E-X and E-Y are employed or the substitution is carried out sequentially.

Alkyl radicals for R¹ which may be mentioned are branched C₁-C₈-alkyl chains, preferably methyl, ethyl, isopropyl, n-propyl, 1-methylethyl, n-butyl, 2-butyl, sec-butyl, tert-butyl, 1-methylpropyl, 2-methylpropyl, 1,1-dimethylethyl, n-pentyl, 2-pentyl, 1-methylbutyl, 2-methylbutyl, 3-methylbutyl, 2,2-dimethylpropyl, l-ethylpropyl, n-hexyl, 2-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 2-ethylhexyl, n-octyl.

Aryl radicals which may be mentioned are phenyl, naphthyl and the substituted phenyl radicals R¹-C₆H₄, where R¹ has the meanings indicated above. In this connection, the alkyl radical R¹ can be located in the o-, m- or p-position.

Solvents

Suitable solvents are all solvents which are inert under the reaction conditions. Preferred solvents are tetrahydrofuran (THF), dioxane and C₁-C₆-ethers, single or mixed C₁-C₆-ethers, C₄-C₁₂-hydrocarbons, such as n-butane, isobutane (2-methylpropane), n-pentane, isopentane (2-methylbutane), neopentane (2,2-dimethylpropane), hexane, 2,2-dimethylbutane, 2-methylpentane, 3-methylpentane, n-heptane, n-octane, n-nonane, n-undecane, n-dodecane, and mixtures of the solvents mentioned.

Bases

Bases which can be employed are organoalkali metal compounds. The use of organolithium compounds, such as n-butyllithium, sec-butyllithium, t-butyllithium and methyllithium, is particularly preferred. The process according to the invention additionally includes the discovery that for the synthesis of the monosubstituted derivatives the base is added in an equimolar amount up to at most in a 1.8 molar excess. For the synthesis of the disubstituted derivatives, the addition of 2 to 4 equivalents of base is necessary.

Amines

In a preferred embodiment of the present invention, the reaction is carried out in the presence of an amine, in particular of a tertiary diamine. Suitable amines which may be mentioned are tetramethylethylenediamine, tetraethylethylenediamine, tetra-n-propylethylenediamine, tetramethylpropylenediamine, tetraethylpropylenediamine, tetra-n-propylpropylenediamine. Chelating amines, such as tetramethylethylenediamine and tetraethylethylenediamine, in particular tetramethylethylenediamine, are particularly suitable.

Process

Customarily, the strong base is introduced in a solvent which is inert under the reaction conditions. The starting material 4-(trifluoromethyl)phenyl 2-tetrahydropyranyl ether, which can be dissolved in, for example, tetrahydrofuran or can be employed directly as a melt, is added thereto. The addition of the electrophile then takes place. This can be present in solid state or as a solution. Alternatively, it is possible to introduce the electrophile and to add the organolithium compound. In the synthesis of the disubstituted derivatives, it has proven advantageous to introduce the base and the ether and then to add the electrophile.

The molar ratio of starting material to electrophile depends on the desired compounds and, in the case of the synthesis of the monosubstituted derivatives, is as a rule between 1:1 and 1:1.9, in the case of the disubstituted derivatives as a rule between 1:2 and 1:4.

The reaction is carried out at −100 to +100° C., preferably at temperatures between −20° C. and +20° C. As a rule, the reaction is carried out in a pressure range from 0.0001 to 200 bar, in particular 0.001 to 20 bar, preferably from 0.001 to 6 bar.

If an amine is employed, this is customarily introduced in the solvent together with the base. The preferred molar ratio of the amine to the organolithium compound is 0:1 to 10:1, in particular 0:1 to 5:1. 0:1, 1:1 and 2:1 are very particularly preferred.

The course of the reaction can be monitored by detection of the starting material 4-(trifluoromethyl)phenyl 2-tetrahydropyranyl ether in the reaction mixture; chromatographic methods such as gas chromatography, HPLC or thin-layer chromatography are suitable for this. As soon as starting material can no longer be detected in the reaction mixture, the reaction is terminated in the customary manner. If desired, the work-up of the reaction mixture can be carried out by methods known to the person skilled in the art, such as distillation, filtration, centrifugation or extraction. The crude products thus obtained can optionally be further purified by means of crystallization, extraction and/or chromatographic methods. The direct use of the reaction mixture for subsequent reactions without further purification is likewise possible, and preferred for the purpose of the present invention.

The 2- or 2,5-substituted derivatives of 4-(trifluoromethyl)-phenol are obtained by removal of the tetrahydropyranyl group of the corresponding 2- or 2,5-substituted derivatives of 4-(trifluoromethyl)phenyl 2-tetrahydropyranyl ether. As a rule, this removal is carried out under acid-catalyzed conditions. Carboxylic acids and mineral acids, for example, are suitable for the acid-catalyzed removal of the tetrahydropyranyl group. Trifluoroacetic acid and hydrochloric acid are particularly preferred.

For the purification of the boronic acids, it can be advantageous to proceed according to the teaching of S. Caron, J. M. Hawkins, J. Org. Chem. (1998) 63 2054.

The process according to the invention can be carried out either batchwise (i.e. “batch process”) or continuously, for example using a reaction tube or a stirred reactor cascade.

The 2- and 2,5-substituted derivatives of 4-(trifluoromethyl)-phenol and of 4-(trifluoromethyl)phenyl 2-tetrahydropyranyl ether prepared by the process according to the invention can be employed as starting materials for the synthesis of dyes, crop protection agents and/or pharmaceuticals.

A further subject of the present invention relates to 2-substituted derivatives of 4-(trifluoromethyl)phenol and/or 2-substituted derivatives of 4-(trifluoromethyl)phenyl 2-tetrahydropyranyl ether of the following formula

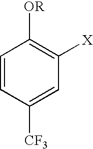

where R is hydrogen or tetrahydropyran-2-yl and if R is hydrogen,

X is B(OH)₂, B(OR¹)₂, B(Oaryl)₂, COR², COOR1, SiR¹₃, Si(aryl)₃, SR², S(aryl), I,

R¹ is C₁-C₈-alkyl,

R² is C₂-C₈-alkyl,

aryl is phenyl, R¹-C₆H₄, if R is tetrahydropyran-2-yl,

X is B(OH)₂, B(OR¹)₂, B(Oaryl)₂, CHO, COR¹, CO(aryl), COOH, COOR¹, SiR¹₃, Si(aryl)₃, OH, SR¹, S(aryl), Cl, Br, I,

R¹ is C₁-C₈-alkyl,

aryl is phenyl, R¹-C₆H₄.

A further subject of the present invention relates to 2,5-disubstituted derivatives of 4-(trifluoromethyl)phenol and/or 2,5-disubstituted derivatives of 4-(trifluoromethyl)phenyl (2-tetrahydropyranyl ether of the following formula

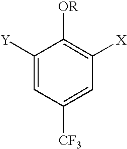

where

R is hydrogen, tetrahydropyran-2-yl,

X is B(OH)₂, B(OR¹)₂, B(Oaryl)₂, B(OR¹)(Oaryl), CHO, COR¹, CO(aryl), COOH, COOR¹, SiR¹_{3,} Si(aryl)₃, OH, SR¹, S(aryl), Cl, Br, I,

Y independently of X is B(OH)₂, B(OR¹)₂, B(Oaryl)₂, B(OR¹)(Oaryl), CHO, COR¹, CO(aryl), COOH, COOR¹, SiR¹₃, Si(aryl)₃, OH, SR¹, S(aryl), Cl, Br, I,

R¹ independently of one another is C₁-C₈-alkyl,

aryl independently of one another is phenyl, naphthyl, R¹-C₆H₄.

### EXAMPLES

Preparation of the starting material: 4-(trifluoromethyl)phenyl 2-tetrahydropyranyl ether

400 g (2.47 mol) of 4-trifluoromethylphenol are introduced in 1 1 of dichloromethane and 525 g (6.25 mol) dihydropyran together with 1 ml of 4-molar hydrogen chloride in dioxane are added dropwise at room temperature. The reaction is markedly exothermic. After stirring overnight, the reaction mixture is washed with 200 ml of sodium hydrogencarbonate solution, and the organic phase is dried over sodium sulfate and concentrated. The residue (618 g, GC: 90 area%, 2.28 mol, yield: 92.3%) is employed in the following step without further purification. It gradually crystallizes completely on standing at room temperature (melting point =38 to 38.7° C.).

### EXAMPLE 1

Preparation of 1-[2-(tetrahydro-2H-pyran-2-yloxy)-5-(trifluoro-methyl) phenyl]-1-propanone (R=2-tetrahydropyranyl, X =C₂H₅CO, Y =H).

24 g (0.207 mol) of tetramethylethylenediamine are introduced at −10° C. and 130 ml of butyllithium in hexane (1.63 molar, 0.212 mol) are added dropwise in about 30 min. After 15 min. the melt of 40 g (GC 90%, 0.146 mol) of 4-(trifluoromethyl)phenyl 2-tetrahydropyranyl ether is added dropwise at −10° C. in the course of 30 min. The lithium complex precipitates in the course of this. After 1 hour, 24 g (0.205 mol) of propionic acid N-methoxy-N-methylamide are added. A turbid solution results, which is added dropwise after 1 hour to 60 ml of 32% strength hydrochloric acid in 40 ml of water. The aqueous phase is separated off and extracted with hexane and the organic phases are concentrated. The residue is recrystallized from hexane at −50° C. 27.3 g (HPLC 96%, 91 mmol, yield 62.3%) of 1-[2-(tetrahydro-2H-pyran-2-yloxy)-5-(trifluoromethyl)phenyl]-1-propanone are obtained in the form of colorless crystals (m.p.: 54 to 56.5° C.).

### EXAMPLE 2

Preparation of 2-hydroxy-5-(trifluoromethyl)benzaldehyde (R =H, X =CHO, Y =H)

241 g (2.08 mol) of tetramethylethylenediamine are introduced at −10° C. and 1.3 1 of butyllithium in hexane (1.63 molar, 2.12 mol) are added dropwise in about 30 min. After 45 min. the melt of 400 g (GC 92%, 1.5 mol) of 4-(trifluoromethyl)phenyl

2-tetrahydropyranyl ether is added dropwise at −10° C. in the course of 30 min. The lithium complex precipitates in the course of this. After 2 hours, 152 g (2.08 mol) of dimethylformamide (DMF) are added dropwise. A turbid solution results which is added dropwise at not more than 45° C. after 15 min. to 750 ml of 38% strength hydrochloric acid in 500 ml of water in the course of 30 min. A strong evolution of gas results. The mixture is stirred overnight, the aqueous phase is separated off, and the organic phase is treated with 70 ml of 4 M dioxane/HCl solution and again stirred overnight. The product is crystallized by cooling to −30° C. (2 to 3 h), filtered off with suction, washed with 500 ml of cold pentane and the product is dried in air. 220 g (GC: 98.3%, 1.13 mol, yield 75%) of 2-hydroxy-5-(trifluoromethyl)benzaldehyde are obtained in the form of colorless crystals (m.p.: 60.1 to 61.8° C.).

### EXAMPLE 3

Preparation of trimethyl[2-(tetrahydro-2H-pyran-2-yloxy)-5-(trifluoromethyl)phenyl]silane (R =2-tetrahydropyranyl, Y =H; X =SiMe₃).

2.9 g (25 mmol) of tetramethylethylenediamine and 15.9 ml (15% strength, 26 mmol) of butyllithium in hexane are introduced at −20° C. and 5 g (HPLC: 98%, 19.9 mmol) of 4-(trifluoromethyl)phenyl 2-tetrahydropyranyl ether dissolved in 10 ml of tetrahydrofuran (THF) are added dropwise. After 30 minutes, 2.63 g (24 mmol) of chlorotrimethylsilane and, after 2 hours, 5 ml of water are added. The reaction mixture is allowed to warm to 20° C., the phases are separated, the organic phase is washed once with saturated sodium chloride solution and the combined aqueous phases are washed three times with 5 ml of methyl t-butyl ether each time. The organic phases are combined, dried over sodium sulfate and concentrated in vacuo. The residue is purified by column chromatography (silica gel, petroleum ether/methyl t-butyl ether =5:1). 5.8 g (HPLC: 98%, 17.9 mmol, yield 90%) of trimethyl[2-(tetrahydro-2H-pyran-2-yloxy) -5-(trifluoromethyl)-phenyl]silane are obtained in the form of a colorless solid. (M.p.: 43 to 46° C.).

### EXAMPLE 4

Preparation of 2-[2-(phenylsulfanyl)-4-(trifluoromethyl)-phenoxy]tetrahydro-2H-pyran (R =2-tetrahydropyranyl, X =S-phenyl, Y =H).

2.9 g (25 mmol) of tetramethylethylenediamine and 15.9 ml (15% strength, 26 mmol) of butyllithium in hexane are introduced at −20° C. and 5 g (HPLC: 98%, 19.9 mmol) of 4-(trifluoromethyl)phenyl 2-tetrahydropyranyl ether dissolved in 10 ml of THF are added dropwise. After 30 minutes, 4.8 g (22 mmol) of diphenyl disulfide and, after 2 hours, 5 ml of water are added.. The reaction mixture is allowed to warm to 20° C., the phases are separated, the organic phase is washed once with saturated sodium chloride solution and the combined aqueous phases are washed three times with 5 ml of methyl t-butyl ether each time. The organic phases are combined, dried over sodium sulfate and concentrated in vacuo. The residue is purified by column chromatography (silica gel, petroleum ether/methyl t-butyl ether =24:1). 6.35 g *(HPLC: 92.3%, 16.6 mmol, yield 82.7%) of 2-[2-(phenylsulfanyl)-4-(trifluoromethyl)phenoxy]tetrahydro-2H-pyran are obtained in the form of a colorless solid. (M.p.: 72 to 73° C.).

### EXAMPLE 5

Preparation of 2-hydroxy-5-(trifluoromethyl)benzoic acid (R =H, X =COOH, Y =H)

2.9 g (25 mmol) of tetramethylethylenediamine and 15.9 ml (15% strength, 26 mmol) of butyllithium in hexane are introduced at −20° C. and 5 g (HPLC: 98%, 19.9 mmol) of 4-(trifluoromethyl)phenyl 2-tetrahydropyranyl ether dissolved in 10 ml of THF are added dropwise. After 30 minutes, carbon dioxide dried by means of a phosphorus pentoxide cartridge is introduced for 30 min. The reaction mixture is allowed to warm to 20° C. overnight, 5 ml of water, 8 ml of conc. hydrochloric acid and 10 ml of methyl t-butyl ether are added and a colorless solid (0.4 g, 4-hydroxybenzotrifluoride-3,5-dicarboxylic acid) is filtered off with suction, the phases are separated, the organic phase is washed once with saturated sodium chloride solution and the combined aqueous phases are washed three times with 5 ml of methyl t-butyl ether each time. The organic phases are combined, dried over sodium sulfate and concentrated in vacuo. The residue is purified by column chromatography (silica gel, petroleum ether/methyl t-butyl ether =95:5, +0.1 ml of AcOH/100 ml of eluent) and extraction under basic conditions with methyl t-butyl ether and under acidic conditions with dichloromethane. 2.2 g (HPLC: 97.8%, 14.5 mmol, yield 52.5%) of 2-hydroxy-5-(trifluoromethyl)benzoic acid are obtained in the form of a colorless solid. (M.p.: 149 to 151° C.).

### EXAMPLE 6

Preparation of 4-(trifluoromethyl)-1,2-benzenediol (R =H, X =OH, Y =H)

15.9 ml (15% strength, 26 mmol) of butyllithium in hexane are introduced at −20° C. and 5 g (HPLC: 98%, 19.9 mmol) of 4-(trifluoromethyl)phenyl 2-tetrahydropyranyl ether dissolved in 40 ml of THF are added dropwise. After 30 minutes, a mixture of 2.7 g (25 mmol) of trimethyl borate and 1.62 g (50% strength, 12 mmol) of boron trifluoride in diethyl ether are added at −70° C. and, after 1.5 hours, 2.3 g (30% strength, 20 mmol) of hydrogen peroxide are added. The reaction mixture is allowed to warm to 20° C., 10 ml of dilute sodium dithionite solution are added, the phases are separated, the organic phase is washed once with saturated sodium chloride solution and the combined aqueous phases are washed twice with 5 ml of THF each time. The organic phases are combined, dried over sodium sulfate and concentrated in vacuo. The residue is purified by column chromatography (silica gel, petroleum ether/methyl t-butyl ether =5:1). 2.2 g (HPLC: 86.1%, 10.6 mmol, yield 53%) of 4-(trifluoromethyl)-1,2-benzenediol are obtained in the form of a red-brown oil.

### EXAMPLE 7

Preparation of 2-[2-chloro-4-(trifluoromethyl)phenoxy]tetrahydro-2H-pyran

(R =2-tetrahydropyranyl, X =Cl, Y =H)

14.7 ml (15% strength, 24 mmol) of butyllithium in hexane are introduced at −70° C. and 5 g (HPLC: 98%, 19.9 mmol) of 4-(trifluoromethyl)phenyl 2-tetrahydropyranyl ether dissolved in 40 ml of THF are added dropwise. After 30 minutes, a solution of 9.5 g of hexachloroethane in 20 ml of THF is added at −70° C. After 4 hours, the reaction mixture is allowed to warm to 20° C. and is concentrated on the next day. The residue is taken up in petroleum ether, the insoluble salts are separated off and the mother liquor is purified by column chromatography (silica gel, petroleum ether/methyl t-butyl ether =9:1) after concentration. 5.2 g (HPLC: 95%, 17.6 mmol, 88.7%) of 2-[2-chloro-4-(trifluoromethyl)phenoxy]tetrahydro-2H-pyran are obtained in the form of a colorless oil.

### EXAMPLE 8

Preparation of 2-iodo-4-(trifluoromethyl)phenyl tetrahydro-2H-pyran-2-yl ether

(R =2-tetrahydropyranyl, X =I, Y =H) 12.2 ml (15% strength, 20 mmol) of butyllithium in hexane are introduced at −70° C. and 5 g (HPLC: 98%, 19.9 mmol) of 4-(trifluoromethyl)phenyl 2-tetrahydropyranyl ether dissolved in 40 ml of THF are added dropwise. After 30 minutes, a solution of 6.3 g (52.5 mmol) of iodine, 7.5 ml of butyllithium in hexane and 19 ml of THF are added at −70° C. After 4 hours, the reaction mixture is allowed to warm to 20° C. and is concentrated. The residue is purified by column chromatography (silica gel, petroleum ether/methyl t-butyl ether =9:1). 5.22 g (HPLC: 94.4% strength, 13.2 mmol, 66.6%) of 2-iodo-4-(trifluoromethyl)phenyl tetrahydro-2H-pyran-2-yl ether are obtained in the form of a colorless oil.

### EXAMPLE 9

Preparation of 2-(1,3,6,2-dioxazaborocan-2-yl)-4-(trifluoro-methyl)phenyl tetrahydro-2H-pyran-2-yl ether (R =2-tetrahydropyranyl, X =B(OR)₂, Y =H)

5 g (HPLC: 98%, 19.9 mmol) of 4-(trifluoromethyl)phenyl 2-tetrahydropyranyl ether are introduced dissolved in 100 ml of THF and 27 ml (15% strength, 44 mmol) of butyllithium in hexane are added dropwise at −70° C. After 45 minutes, this solution is slowly added dropwise to a mixture of 15.04 g (80 mmol) of triisopropyl borate and 40 ml of THF at −70° C. After 1.5 hours, the reaction mixture is allowed to warm to 20° C. and is concentrated on a rotary evaporator. The residue is taken up in 100 ml of toluene, the insoluble constituents are separated off, the mother liquor is concentrated to 50 ml and the residue is mixed with 2.1 g (20 mmol) of diethanolamine. The product crystallizes after a short time. The crystal magma is taken up in 50 ml of MTBE, and the crystals are filtered off with suction, washed with MTBE and dried. 4.9 g (HPLC: 98.4%, 13.4 mmol, 67.5%) of 2-(1,3,6,2-dioxazaborocan-2-yl)-4-(trifluoromethyl)phenyl tetrahydro-2H-pyran-2-yl ether are obtained in the form of a colorless solid. (M.p.: 192° C.).

We claim:

1. A process for the preparation of compounds of the general formula 1 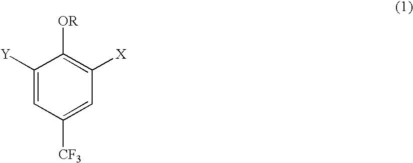
where
R is hydrogen, tetrahydropyran-2-yl,
X is B(OH)₂, B(OR¹)₂, B(Oaryl)₂, B(OR¹)(Oaryl), CHO, COR¹, CO(aryl), COOH, COOR¹, SiR¹₃, Si(aryl)₃, OH, SR¹, S(aryl), Cl, Br, I,
Y is H, B(OH)₂, B(OR¹)₂, B(Oaryl)₂, B(OR¹)(Oaryl), CHO, COR¹, CO(aryl), COOH, COOR¹, SiR¹₃, Si(aryl)₃, OH, SR¹, S(aryl), Cl, Br, I,
R¹ independently of one another is C₁-C₈-alkyl, aryl independently of one another is phenyl, naphthyl, R¹-C₆H₄, R¹O-C₆H₄,
which comprises reacting the compound of the formula 2 4-(trifluoromethyl)phenyl 2-tetrahydropyranyl ether 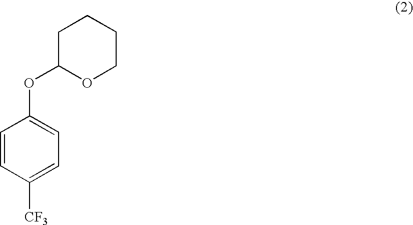
in the presence of a base with an electrophile E-X or a combination of electrophiles E-X and E-Y, where X and Y have the meanings indicated above.

2. A process as claimed in claim 1, wherein the base employed is an organolithium compound.

3. A process as claimed in at least one of the abovementioned claims, wherein the reaction is carried out in the presence of an amine.

4. A process as claimed in claim 3, wherein a chelating amine is employed.

5. A process as claimed in at least one of the aforementioned claims, wherein the reaction is carried out at a temperature of −100 to +100° C.

6. A process as claimed in at least one of the aforementioned claims, wherein the reaction is carried out at a pressure of 1 to 200 bar.

7. A 2-substituted derivative of 4-(trifluoromethyl)phenol and of the 4-(2-trifluoromethyl)phenyl 2-tetrahydropyranyl ether of the following formula 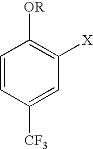
where R is hydrogen or tetrahydropyran-2-yl and if R is hydrogen,
X is B(OH)₂, B(OR¹)₂, B(Oaryl)₂, COOR¹, SiR¹₃, Si(aryl)₃, SR², S(aryl), I,
R¹ independently of one another is C₁-C₈-alkyl,
R² independently of one another is C₂-C₈-alkyl, aryl is phenyl, naphthyl, R¹-C₆H₄, R¹O-C₆H₄,
if R is tetrahydropyran-2-yl,
X is B(OH)₂, B(OR¹)₂, B(Oaryl)₂, CHO, COR¹, CO(aryl), COOH, COOR¹, SiR¹₃, Si(aryl)₃, OH, SR¹, S(aryl), Cl, Br, I,
R¹ independently of one another is C₁-C₈-alkyl, aryl independently of one another is phenyl, naphthyl, R¹-C₆H₄, R¹O-C₆H₄.

8. A 2,5-disubstituted derivative of 4-(trifluoromethyl)phenol and of the 4-(2-trifluoromethyl)phenyl 2-tetrahydropyranyl ether of the following formula 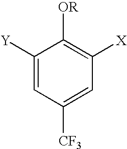
where
R is hydrogen, tetrahydropyran-2-yl,
X is B(OH)₂, B(OR¹)₂, B(Oaryl)₂, B(OR¹)(Oaryl), CHO, COR¹, CO(aryl), COOH, COOR¹, SiR¹₃, Si(aryl)₃, OH, SR¹, S(aryl), Cl, Br, I,
Y independently of X is B(OH)₂, B(OR¹)₂, B(Oaryl)₂, B(OR¹)(Oaryl), CHO, COR¹, CO(aryl), COOH, COOR¹, SiR¹₃, Si(aryl)₃, OH, SR¹, S(aryl), Cl, Br, I,
R¹ independently of one another is C₁-C₈-alkyl, aryl independently of one another is phenyl, naphthyl, R¹-C₆H₄, R¹O-C₆H₄.
